(19) United States
(12) Patent Application Publication
(10) Pub. No.: US 2026/0363936 A1
(43) Pub. Date: Nov. 19, 2026

(54) MOVEMENT FUNCTION IMPROVEMENT DEVICE AND MOVEMENT FUNCTION IMPROVEMENT METHOD

(71) Applicant: CYBERDYNE Inc., Tsukuba-shi, Ibaraki (JP)

(72) Inventor: Yoshiyuki SANKAI, Tsukuba-shi, Ibaraki (JP)

(21) Appl. No.: 19/121,101

(22) PCT Filed: Aug. 25, 2023
(86) PCT No.: PCT/JP2023/030629

§ 371 Date: Apr. 14, 2025

(30) Foreign Application Priority Data
Oct. 31, 2022 (JP) 2022-175158

(51) Int. Cl. A61H 1/02; A61H 3/00

(52) U.S. Cl. CPC A61H 1/0262; A61H 3/00; A61H 2201/164; A61H 2201/165; A61H 2201/5061; A61H 2201/5069; A61H 2230/605

(57) ABSTRACT

To minimize the difference between a subject's intention to move and a kinetic phenomenon, the above-described difference is corrected by a movement command from their cranial nervous system and, at the same time, a synthesized control state is feedback-adjusted on the basis of biofeedback loops which are promoted interactively between the subject's body and a movement mechanism unit.

### CROSS-REFERENCE TO RELATED APPLICATIONS

This application is a U.S. national stage application of International Application No. PCT/JP 2023/030629, filed Aug. 25, 2023, which claims benefit of priority from JP 2022-175158, filed Oct. 31, 2022, the contents of which are incorporated herein by reference in its entirety for all purposes.

### TECHNICAL FIELD

The present invention relates to a movement function improvement device and a movement function improvement method and is particularly suited for application to medical treatments and rehabilitation of a subject who has a functional disorder of their cranial nervous system or body system.

### BACKGROUND ART

In recent years, the development of various devices for assisting or substituting movements of a subject who has motor function impairments has been underway. For example, wearable motion assistance devices capable of controlling and assisting movements based on a bioelectrical potential caused by voluntary muscle activities according to the subject's intention are widely used (for example, see PTL 1) as the above-described devices.

Such wearable motion assistance devices can not only assist movements according to the subject's intention, but also can be applied to, for example, therapeutic treatment and rehabilitation which are aimed at recovery of the motor functions. Particularly, many cases are reported where subjects who has diseases in their brains, nerves, and muscular systems experienced functional improvements by making use of the above-described wearable motion assistance device.

When the wearable motion assistance device functions and moves the subject's body, whose motor functions became defective, based on a signal from the subject's own cranial nervous system, this results in making the subject move their musculoskeletal system with their own intention, information of their sensory system flows through inside and outside the human body to the cranial nervous system, thereby establishing bidirectional biofeedback loops between the cranial nervous system and the musculoskeletal system.

By repeating this, it is believed that synaptic connections of the brain, the nerves, and the muscular system are enhanced, relearning and functional regeneration progresses, and improvements of the bodily functions of the subject who has the disease in their brain, nervous system, or muscular system are promoted (for example, see NPL 1).

### CITATION LIST

### Patent Literature

PTL 1: Japanese Patent No. 4178185

### Non-Patent Literature

NPL 1: Yoshiyuki Sankai, Hiroshi Tanaka, “Approved Robotic Medical Equipment for Improving Lower Limb Motor Function,” the 14th New Machine Promotion Award Winners' Achievement Summary, 2016, pp. 5-8

### SUMMARY OF THE INVENTION

### Problems to be Solved by the Invention

Meanwhile, a human realizes voluntary movements based on their intention to move while adaptively adjusting movement patterns to cooperatively activate a plurality of muscles such as flexor muscles and extensor muscles; however, how the adjustment mechanism works to cause such various muscle groups to move cooperatively so that the aimed movement can be realized has been poorly clarified.

Medically, experimental studies are carried out in order to elucidate information processing focused on brains, for example, by using model animals to explore how the cranial nervous system generates a specific movement pattern, and by conducting analytical research (connectomics) by means of a connectome (a comprehensive map for connections within the nervous system) to explore via what kind of circuit structures the nerve cells control movements.

However, the experimental study to elucidate the information processing focused on the brain cannot deal with the motor functions of a body, so it is difficult to elucidate cooperative voluntary movements.

Moreover, even if the experimental study about not only the brain, but also the body system is conducted, and if the aforementioned wearable motion assistance device is not used, it is believed to be very difficult to prove that when the subject performs a specific voluntary movement, the bodily functions for the voluntary movement are improved and reconstructed as a result of transmissions and receptions of a voluntary movement command signal for moving the body parts and a sensory signal, which is generated as a result of successful movements, between the central system (the brain and the spinal cord) and the peripheral system (the motor nerves, the muscular system, and the sensory nerves).

The present invention was devised in consideration of the above-described circumstances and proposes a movement function improvement device and a movement function improvement method which are capable of improving the motor functions of the subject's brain, nerves, and muscular system by repeated voluntary movements by the subject.

### Means to Solve the Problems

In order to solve the above-described problems, the present invention includes: a movement mechanism unit that is used in a manner united with a subject and has a drive unit to be driven actively or passively in conjunction with body movements of the subject; a signal detection unit that detects changes of an ionic current transmitted from a cranial nervous system to a muscular system of the subject as a biopotential signal appearing on a skin surface; a periarticular detection unit that detects periarticular physical quantities caused by body movements of the subject based on an output signal from the drive unit; a voluntary control unit that controls the drive unit to cause the subject's intention to move to be reflected in a kinetic phenomenon based on the biopotential signal and the periarticular physical quantities; an autonomous control unit that stores respective reference parameters of phases which are a series of minimum movement units constituting the subject's movement patterns classified as tasks, estimates a phase of the subject's task by comparing the periarticular physical quantities with the reference parameters stored in the data storage unit, and controls the drive unit to generate motive power according to the phase; and a synthetic control unit that stores a control ratio of the voluntary control unit and the autonomous control unit, which is set for each phase of each task, in the data storage unit and synthesizes control states by the voluntary control unit and the autonomous control unit so as to achieve the control ratio according to the relevant phase, wherein the synthetic control unit: compensates for physical impedance of an entire system, which is composed of the entire device and the subject, based on the periarticular physical quantities in accordance with physical properties and gravity of the entire system including human body properties of the subject; and corrects a difference between the subject's intention to move and the kinetic phenomenon by a movement command from the cranial nervous system and, at the same time, feedback-adjusts the synthesized control state on the basis of biofeedback loops interactively promoted between the subject's body and the movement mechanism unit in order to minimize the difference between the subject's intention to move and the kinetic phenomenon.

As a result, when the subject repeatedly performs the voluntary movements by using the movement mechanism unit, the movement function improvement device can improve the motor functions of the subject's brain, nerves, and muscular system in conjunction with the correction of the difference between the movement command from the cranial nervous system and the actual kinetic phenomenon.

Moreover, the present invention includes: a gravity center position detection unit that detects a gravity center position of the subject based on a load change on foot sole surfaces of the subject; and a walking state recognition unit that recognizes a walking state of the subject based on the control state by the synthetic control unit and the gravity center position by the gravity center position detection unit, wherein the voluntary control unit controls the drive unit to cause the subject's intention to move to be reflected in the kinetic phenomenon on the basis of the biopotential signal, the periarticular physical quantities, and the walking state recognized by walking state recognition unit.

As a result, even if it is difficult for the subject to move the lower limb by themselves, the movement function improvement device makes it possible to perform the walking movement safely; and at the same time, when the voluntary control unit controls the drive unit, it is possible to improve the accuracy of the kinetic phenomenon in which the subject's intention to move is reflected.

Furthermore, in the present invention, when forming the biofeedback loops, a minimum motion control unit for realizing voluntary movements caused by the subject's intention is set as a minimum voluntary motor control unit composed of the cranial nervous system, synaptic connections, and the muscular system; and the biofeedback loops are established for each minimum voluntary motor control unit that forms body movements which are performed cooperatively to realize the kinetic phenomenon by using the movement mechanism unit.

As a result, with the movement function improvement device, by explicitly incorporating the minimum voluntary motor control unit, in which the influences of the diseased area and the disease cause are reflected, into the process of the functional improvement and treatment based on the basic theory of the biofeedback loops by the movement mechanism unit, other minimum voluntary motor control units are also influenced and synchronize with the movements of the movement mechanism unit, which causes their cooperative movements to progress; and for the purpose of realization of the aimed movement, the functions of the respective minimum voluntary motor control units synchronize with the movements of the movement mechanism unit and are enhanced and adjusted, thereby making it possible to achieve the functional improvement of the brain, the nerves, and the muscular system.

Furthermore, in the present invention, the periarticular detection unit detects, as the periarticular physical quantities, an absolute angle, a rotation angle, an angular velocity, angular acceleration, and a driving torque between a rotator-side frame and a stator-side frame of the drive unit for a movement assisting tool.

Furthermore, in a state where a movement mechanism unit having a drive unit to be driven actively or passively in conjunction with body movements of a subject is used in a manner united with the subject, the present invention includes: a voluntary control step of controlling the drive unit to cause the subject's intention to move to be reflected in a kinetic phenomenon based on a biopotential signal, which indicates changes of an ionic current transmitted from a cranial nervous system to a muscular system of the subject and appears on a skin surface, and periarticular physical quantities which are caused by body movements of the subject and are detected based on an output signal from the drive unit; an autonomous control step of storing respective reference parameters of phases which are a series of minimum movement units constituting the subject's movement patterns classified as tasks, estimating a phase of the subject's task by comparing the periarticular physical quantities with the reference parameters stored in the data storage unit, and controlling the drive unit to generate motive power according to the phase; and a synthetic control step of storing a control ratio of the voluntary control step and the autonomous control step, which is set for each phase of each task, in the data storage unit and synthesizing control states by the voluntary control step and the autonomous control step so as to achieve the control ratio according to the relevant phase, wherein in the synthetic control step, physical impedance of an entire system, which is composed of the entire device and the subject, is compensated for based on the periarticular physical quantities in accordance with physical properties and gravity of the entire system including human body properties of the subject; and a difference between the subject's intention to move and the kinetic phenomenon is corrected by a movement command from the cranial nervous system and, at the same time, the synthesized control state is feedback-adjusted on the basis of biofeedback loops interactively promoted between the subject's body and the movement mechanism unit in order to minimize the difference between the subject's intention to move and the kinetic phenomenon.

As a result, when the subject repeatedly performs the voluntary movements by using the movement mechanism unit, the movement function improvement method can improve the motor functions of the subject's brain, nerves, and muscular system in conjunction with the correction of the difference between the movement command from the cranial nervous system and the actual kinetic phenomenon.

### Advantageous Effects of the Invention

According to the present invention, it is possible to implement the movement function improvement device and the movement function improvement method which are capable of improving the motor functions of the subject's brain, nerves, and muscular system in connection with the correction of the difference between the movement command from the cranial nervous system and the actual kinetic phenomenon when the subject repeats performing the voluntary movements by using the movement mechanism unit.

### BRIEF DESCRIPTION OF DRAWINGS

FIG. 1 is a conceptual diagram for explaining a basic theory for biofeedback loops according to the present invention;

FIG. 2 is a conceptual diagram for explaining a minimum voluntary motor control unit(s);

FIG. 3 is a conceptual diagram illustrating a transition status where the basic theory of the aforementioned biofeedback loops is applied to a subject;

FIG. 4 is a schematic diagram illustrating an appearance configuration of a movement function improvement device of a lower limb type according to this embodiment;

FIG. 5 is a block diagram illustrating the configuration of a control system for the movement function improvement device in FIG. 4;

FIG. 6 is a conceptual diagram illustrating an example of respective tasks and respective phases stored in a data storage unit;

FIG. 7 is a diagram illustrating a state where the subject wears this movement function improvement device;

FIG. 8 is a diagram illustrating a state where the subject wears this movement function improvement device;

FIG. 9 is a schematic diagram illustrating an appearance configuration of a movement function improvement device including a movement mechanism unit of a lower back type according to another embodiment;

FIG. 10 is a schematic diagram illustrating a major configuration of the movement function improvement device in FIG. 9;

FIG. 11 is a schematic diagram illustrating moving states and a movable range of the movement function improvement device in FIG. 9;

FIG. 12 is a schematic diagram illustrating an appearance configuration of a movement function improvement device including a movement mechanism unit of a single joint type according to another embodiment;

FIG. 13 is a schematic diagram illustrating a state where the movement function improvement device in FIG. 12 is applied to the subject's right leg knee joint; and

FIG. 14 is a schematic diagram illustrating a state where the movement function improvement device in FIG. 12 is applied to the subject's left arm elbow joint.

### DESCRIPTION OF EMBODIMENTS

An embodiment of the present invention will be described below in detail with reference to the drawings.

### (1) Basic Theory of Biofeedback Loops in The Present Invention

Medically, the experimental studies are conducted to elucidate the information processing focused on brains as the means for improving the motor functions of the subject's brain, nerves, and muscular system. However, with just the brain study which is separated from the motor system, it is difficult to target at bodily motor functions.

The motor system of a human body is configured of central nerves which are the brain and the spinal cord, peripheral nerves, and a musculoskeletal system; and when dealing with an information flow of the centrifugal nerves away from the central region towards peripheral regions, an information transmission system is formed of these regions which are connected, but appropriate motion control would not be realized with only them.

Specifically speaking, the locations where information of the centrifugal nerves is transmitted are muscle fibers which are involved when muscles contract; however, regarding the information after the contraction, even if the information is transmitted from the brain, passes through the spinal cord, and reaches the muscle fibers via the motor nerves, that information will not return to the brain. So, by just analyzing the “motor unit(s)” which is composed of the motor nerves and the muscle fibers, it is almost impossible to clarify the mechanism of improvements of the motor functions or to create a motion control improvement technology.

Accordingly, there is some study to try achieving functional improvements by using a robot technology and moving the articular system and the muscular system by externally inputting movements to the articular system and the muscular system such as legs and hands; however, it is also reported that the functional improvements are not achieved simply by giving external force to move the articular system and the muscular system such as legs and hands.

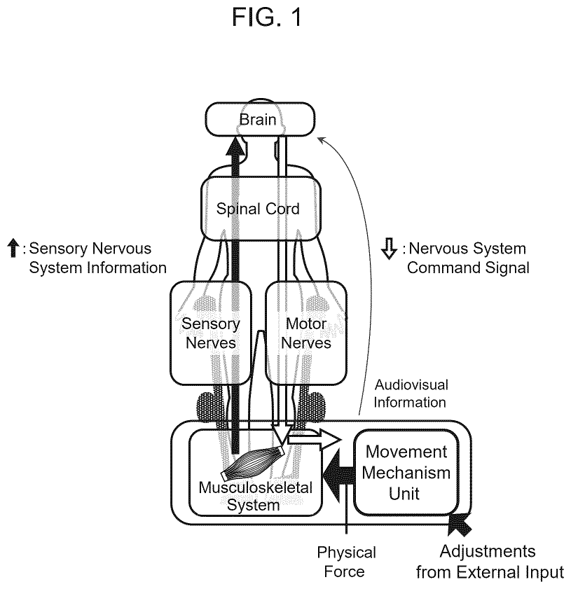

According to the present invention as illustrated in FIG. 1, it is proved that when the subject performs a certain movement by using the movement mechanism unit having the drive unit to be driven actively or passively in conjunction with the subject's body movements, a command signal (a centrifugal nerve signal) to try to move according to the intention to move and a sensory signal (an afferent nerve signal) generated as a result of successful movement are transmitted and received between the central system (the brain and the spinal cord) and the peripheral system (the motor nerves, the muscular system, and the sensory nerves), thereby improving and reconstructing the bodily functions for voluntary movements.

The movement function improvement method according to the present invention includes a voluntary control step of causing a movement(s) according to the subject's intention to move, an autonomous control step of generating preset ideal motive power, and an impedance control step (including gravity compensation control) for alleviating a feeling of difficulty to move due to the load and viscous frictions of the movement mechanism unit itself, so that the subject can feel as if the movement mechanism unit were a part of their body, and functional fusion and integration between the subject and the movement mechanism unit can be realized.

Furthermore, by this movement function improvement method, the synthetic control step of synthesizing the control states by the voluntary control step and the autonomous control step so as to obtain a control ratio according to the phase of the relevant task not only executes the above-described impedance control step, but also corrects the difference between the subject's intention to move and the kinetic phenomenon by a movement command from the cranial nervous system and, at the same time, feedback-adjusts the synthesized control state on the basis of biofeedback loops promoted between the subject's body and the movement mechanism unit.

The above-mentioned biofeedback loops are formed by repeating the following circulating flow of: activation of proprioceptors called muscle spindles and tendon spindles in muscles and tendons in synchronization with muscle contraction as a result of the transmission of nervous system command information from the brain through the spinal cord via the motor nerves to the muscle fibers; feedback of its activation information, as information upon the muscle contraction, via the sensory nerves to the central nervous system (the spinal cord and the brain); and enhancements and adjustments of synaptic connections between the nerves and nerves and between the nerves and the muscles based on the feedback information.

Accordingly, by the movement function improvement method, as the subject repeatedly performs the voluntary movements by using the movement mechanism unit, the motor functions of the subject's brain, nerves, and muscular system can be improved in conjunction with the correction of the difference between the movement command from the cranial nervous system and the actual kinetic phenomenon.

One of features of the present invention is that, when forming the biofeedback loops, a minimum voluntary motor control unit which is composed of the cranial nervous system (the brain and the spinal cord), synaptic connections (the synaptic connections between the nerves and nerves and the synaptic connections between the nerves and the muscles), the muscular system (such as muscle fibers (extrafusal muscle fibers to bind with a motor neurons and intrafusal muscle fibers to bind with y motor neurons) and tendon fibers, muscle spindles, and tendon spindles) can be configured as a minimum motion control unit for realizing the voluntary movement(s), which is caused by a person's intention, to establish the biofeedback loops.

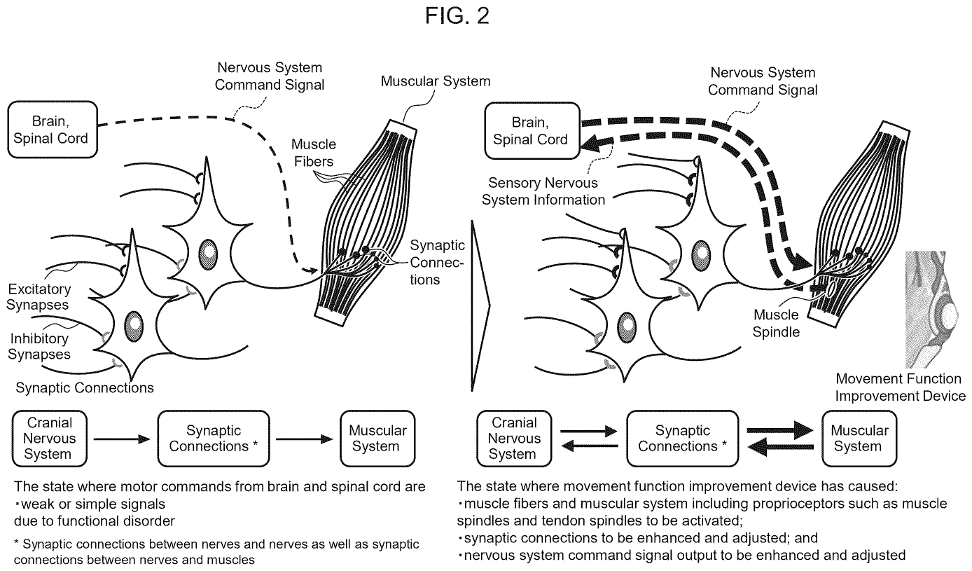

Specifically speaking, the minimum voluntary motor control unit is a control unit for realizing a minimum voluntary movement which is formed in a path formed of the cranial nervous system (the brain, the spinal cord, the motor nerves), the muscle fibers, movements (reactions), the muscle spindles and the tendon spindles, and the cranial nervous system (the sensory nerves, the spinal cord nerves, and the brain) as illustrated in FIG. 2.

As the minimum voluntary motor control unit which forms a specific joint movement cooperatively work with other joint movements, the above-mentioned minimum voluntary motor control unit is formed in plurality together with other minimum voluntary motor control units which are related to the above-mentioned minimum voluntary motor control unit, thereby realizing the targeted overall movements.

Under this circumstance, the synaptic connections between the nerves and the nerves and between the nerves and the muscles in the minimum voluntary motor control units are respectively adjusted and enhanced in a group of the minimum voluntary motor control units regarding the targeted overall movements and in the entire adjustment system, for example, balance adjustments of the posture which are performed unconsciously.

Furthermore, by using the movement mechanism unit (such as a lower limb type, a single joint type, a lower back type, a hand type, and a finger type as described later) in joint movements driven by muscle groups (so-called flexor-type and extensor-type muscle groups) which are configured of the minimum voluntary motor control units (units), and in more complex and cooperative movements configured of the respective articular systems, it becomes possible to make it respond to the minimum units to a higher-level, complex body system and the functional improvements of the brain, the nerves, and the muscular system can be realized.

Practically, when the minimum voluntary motor control units are recognized as units for promoting synaptic plasticity, neural plasticity, and muscular plasticity which are basic nerve functions to improve the functions of the brain, the nerves, and the muscular system, the process for activating the subject's self-healing power according to diseases or symptoms (such as relaxation, rigidity, trembling, stiffness, ataxia, and simultaneous contraction) varies depending on each relevant minimum voluntary motor control unit.

Therefore, unit constituent elements of the minimum voluntary motor control unit for improving the motor functions of the brain, the nerves, and the muscular system vary depending on each disease or each symptom and the relevant sites and the relevant range of other minimum voluntary motor control units with which the relevant minimum voluntary motor control unit is involved vary, so that it becomes possible to construct treatment control strategies by using them as minimum units when performing various kinds of adjustments of the movement mechanism unit according to the subject's condition (such as tuning of parameters for realizing the movement(s) according to the subject's intention to move and establishing the biofeedback loops).

The movement function improvement method according to the present invention improves the functions of the respective minimum voluntary motor control units which constitute the targeted overall voluntary movements by detecting signals which originate from the nervous system that cooperatively works with the voluntary intention, and which are obtained from a path influenced by a diseased area and a disease cause, and by focusing attention on the minimum voluntary motor control units relating to that path.

In order to realize a human's voluntary movements, the appearance of their intention to move voluntarily at the cerebrum is the base point and a nervous system signal of that movement intention is transmitted from the brain to the spinal cord, the motor nerves, and the muscle fibers and the target movement is realized eventually. However, this flow describes the flow from the appearance of the intention to the generation of the movement too roughly, so it is difficult to explicitly recognize the influences of the diseased area and the disease cause on the voluntary movements from the brain and the nervous system through the synaptic connections to the muscular system including the sensory nerves of the muscle fibers and the muscle spindles, and y loops by y motor neurons, and it is also difficult to explicitly perform a treatment in consideration of the influences of the diseased area and the disease cause. Accordingly, just the repetition of simple movements as training is the only thing which can be done in the actual clinical environment.

Therefore, by explicitly incorporating the minimum voluntary motor control unit, in which the influences of the diseased area and the disease cause are reflected, into the process of the functional improvement and treatment based on the basic theory of the biofeedback loops by the movement mechanism unit, other minimum voluntary motor control units are also influenced and synchronize with the movements of the movement mechanism unit, which causes their cooperative movements to progress; and for the purpose of realization of the aimed movement, the respective minimum voluntary motor control units synchronize with the movements of the movement mechanism unit and the functions of their unit constituent elements are enhanced and adjusted, thereby making it possible to achieve the functional improvement of the brain, the nerves, and the muscular system as a new method different from conventional methods.

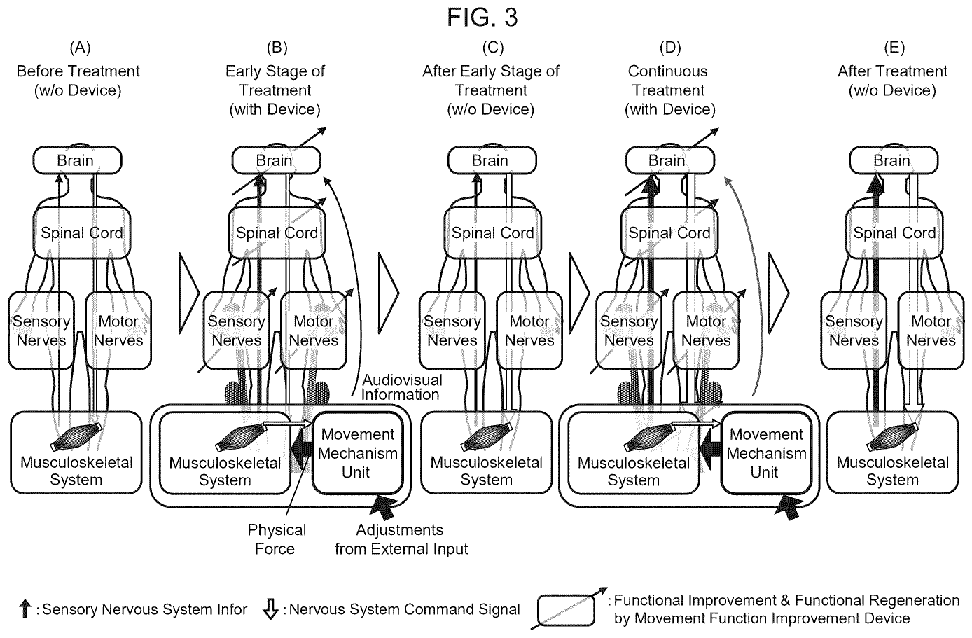

FIG. 3(A) to 3(E) illustrate a transition status where the basic theory of the aforementioned biofeedback loops is applied to the subject. The transition starts from the state of not wearing the movement mechanism unit before the treatment (FIG. 3(A)); and in an early stage of the treatment, sensory nervous system information from the musculoskeletal system by joint movements by using the movement mechanism unit is fed back to the central nervous system (the brain and the spinal cord) (FIG. 3(B)).

Subsequently, in the state of not wearing the movement mechanism unit after the early stage of treatment (FIG. 3(C)), a sense of feedback of the sensory nervous system information slightly remains; however, by performing the treatment continuously by using the movement mechanism unit (FIG. 3(D)), the difference between the subject's intention to move and the kinetic phenomenon is corrected by a movement command from the central nervous system on the basis of the biofeedback loops promoted interactively between the subject's body and the movement mechanism unit.

Then, even in the state of not wearing the movement mechanism unit after the continuous treatment, the motor functions of the subject's brain, nerves, and muscular system can be improved, while activating the subject's self-healing power in conjunction with the correction of the difference between the movement command from the central nervous system and the actual kinetic phenomenon (FIG. 3(E)).

### (2) Movement Function Improvement Device in This Embodiment

### (2-1) Configuration of Movement Mechanism Unit (Lower Limb Type: Hardware)

An explanation will be provided about the configuration of the movement function improvement device including a movement mechanism unit for a lower body (lower limb type) according to this embodiment.

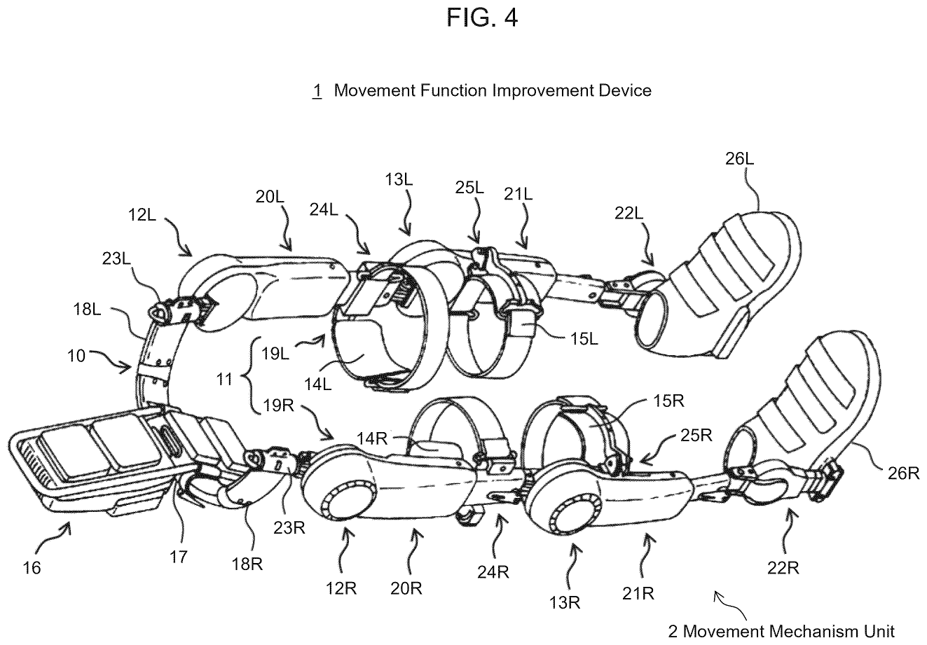

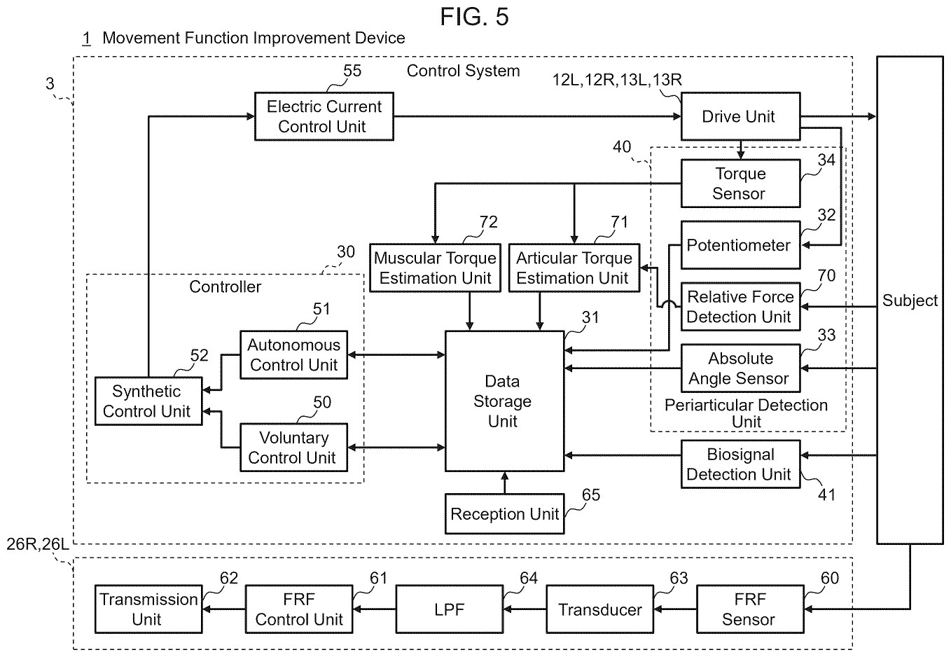

Referring to FIG. 4, the movement function improvement device 1 is used in a manner united with the subject and is configured so that a control system 3 (FIG. 5) having a functional configuration of the voluntary control step, the autonomous control step, the impedance control step, and the basic theory of the biofeedback loops which are described earlier is installed in a movement mechanism unit 2 having a drive mechanism to be driven actively or passively in conjunction with the subject's body movements.

The movement function improvement device 1 is a device which applies a driving force by autonomous control according to respective walking phases constituting the subject's walking movements and applies a driving force by voluntary control based on a biopotential signal; and detects the biopotential signal and movement angles of the subject's hip joints and knee joints and operates to apply the driving force from the drive mechanism based on this detected signal.

The movement mechanism unit 2 is composed of: a lower-back frame 10 to be mounted on the subject's lower back; a lower limb frame 11 to be mounted on the subject's lower limbs; a plurality of drive units (drive mechanism) 12L, 12R, 13L, 13R provided at the lower limb frame 11 corresponding to the subject's joints; and cuffs 14L, 14R, 15L, 15R as assist force applying members attached to the lower limb frame 11 so as to cause the forces of the drive units 12L, 12R, 13L, 13R to act on the subject from the front or the back.

The movement function improvement device 1 has, in addition to the aforementioned movement mechanism unit 2, a controller 30 (FIG. 5 described later) for controlling the drive units (the drive mechanism) 12L, 12R, 13L, 13R according to signals attributable to the subject's lower limb movements, a back side unit 16 in which the controller 30 is installed, and an operation unit (which is not illustrated in the drawing) to be used by a helper.

The controller 30 (FIG. 5) can drive the lower limb frame 11 relatively around output axes of actuators for the drive units 12L, 12R, 13L, 13R corresponding to the subject's joints. Each drive unit 12L, 12R, 13L, 13R is equipped with a sensor group for detecting a driving torque, a rotation angle, etc. of its actuator. Incidentally, the back side unit 16 is equipped with a battery unit (which is not illustrated in the drawing) for supplying a driver power source of the entire device.

The lower-back frame 10 has: a rear lower-back frame part 17 that is a member of a substantially C-shaped in planar view, which is open forwards and is capable of receiving the subject's lower back and surrounding the subject's lower back from its rear part to its right and left side parts and which can be positioned at the subject's back; and a left lower-back frame part 18L and a right lower-back frame part 18R which bend from both ends of the rear lower-back frame part 17 and extend forwards.

The left lower-back frame part 18L and the right lower-back frame part 18R are connected via an opening degree adjustment mechanism (which is not illustrated in the drawing) to the rear lower-back frame part 17. Base parts of the left lower-back frame part 18L and the right lower-back frame part 18R are inserted into and retained by the rear lower-back frame part 17 so that they can slide to the right and left directions.

The lower limb frame 11 has a right lower-limb frame 19R to be mounted on the subject's right lower limb and a left lower-limb frame 19L to be mounted on the subject's left lower limb. The left lower-limb frame 19L and the right lower-limb frame 19R are formed to be bilaterally symmetrical to each other.

The left lower-limb frame 19L has a left thigh frame 20L to be positioned on the left side of the subject's left thigh, a left lower leg frame 21L to be positioned on the left side of the subject's left lower leg, and a left leg lower-end frame 22L on which the back of the subject's left leg (when the subject wears shoes, the bottom of the left-side shoe) is to be mounted. The left lower-limb frame 19L is coupled to a top end part of the left lower-back frame part 18L via a lower-back part connecting mechanism 23L.

The right lower-limb frame 19R has a right thigh frame 20R to be positioned on the right side of the subject's right thigh, a right lower leg frame 21R to be positioned on the right side of the subject's right lower leg, and a right leg lower-end frame 22R on which the back of the subject's right leg (when the subject wears shoes, the bottom of the right-Our Reference side shoe) is to be mounted. The right lower-limb frame 21R is coupled to a top end part of the right lower-back frame part 18R via a lower-back part connecting mechanism 23R.

Incidentally, the lower-back frame 10 (the rear lower-back frame 17, the right lower-back frame 18R, and the left lower-back frame 18L) and the lower limb frame 11 (the right lower-limb frame 19R and the left lower-limb frame 19L) have a frame body formed in a long narrow plate shape made of, for example, metals such as stainless steel or carbon fibers, so the frame body is formed to be lightweighted and have high rigidity. In this embodiment, carbon fiber reinforced plastic (CFRP) and extra super duralumin which is an aluminum alloy are used as strength members.

Each one of the cuffs 14L, 14R, 15L, 15R is provided on each relevant frame of the left thigh frame 20L, the right thigh frame 20R, the left lower leg frame 21L, and the right lower leg frame 21R.

The cuffs provided on the left thigh frame 20L and the right thigh frame 20R (hereinafter referred to as “thigh cuffs”) 14L, 14R are supported by thigh cuff support mechanisms 24L, 24R attached to lower end parts of the thigh frame bodies. The thigh cuff 14L, 14R has a mounting surface which is bent in an arc shape and can be placed on and in contact with the subject's thigh to make the mounting surface engage with the subject's thigh. A fitting member is attached to the mounting surface of the thigh cuff 14L, 14R so that the mounting surface can be attached firmly to the subject's thigh without any space between them.

The cuffs provided on the left lower leg frame 21L and the right lower leg frame 21R (hereinafter referred to as “lower-leg cuffs”) 15L, 15R are supported by the lower-leg cuff support mechanism 25L, 25R attached to upper end parts of the upper elements. The lower-leg cuff 15L, 15R has a mounting surface which is bent in an arc shape and can be placed on and in contact with the subject's lower leg to make the mounting surface engage with the subject's lower leg. A fitting member is attached to the mounting surface of the lower-leg cuff 15L, 15R so that the mounting surface can be attached firmly to the subject's lower leg without any space between them.

When the subject actually wears this movement function improvement device, dedicated shoes 26L, 26R are mounted on the right and left foot parts, respectively, the lower-leg cuffs 15L, 15R are mounted on the right and left lower leg parts, respectively, and the thigh cuffs 14L, 14R are further mounted on the right and left thigh parts, respectively. Then, the shoes and cuffs are fastened with belts or the like so that these foot parts, lower leg parts, and thigh parts are united with their respectively corresponding frames.

These dedicated shoes 26L, 26R are configured as a pair of right and left shoes and are retained in a state where the subject's toes to their ankle are firmly attached to each shoe; and a load can be measured by a floor reaction force sensor (an FRF sensor 60 described later) provided on the sole of each foot.

Accordingly, the movement function improvement device 1 can control and assist walking movements according to the biopotential signal in conjunction with a voluntary nervous system command signal according to the intention of the subject who wears the movement mechanism unit 2.

### (3) Control System in Movement Function Improvement Device

FIG. 5 is a block diagram illustrating the configuration of the control system 3 for the movement function improvement device 1. Referring to FIG. 5, the control system 3 for the movement function improvement device 1 has: the controller 30 in charge of the overall control of the entire system; a data storage unit 31 in which various kinds of data are formed into a writable/readable database according to commands from the controller 30; and the drive units 12L, 12R, 13L, 13R to be driven actively or passively in conjunction with the subject's lower limb movements.

Moreover, the control system 3 is provided with a periarticular detection unit 40, which has a potentiometer 32, an absolute angle sensor 33, and a torque sensor 34, and detects periarticular physical quantities caused by the subject's body movements based on output signals from the drive units 12L, 12R, 13L, 13R.

The periarticular detection unit 40 detects, as the periarticular physical quantities, an absolute angle, a rotation angle, an angular velocity, angular acceleration, and a driving torque between a rotator-side frame and a stator-side frame of the drive units 12L, 12R, 13L, 13R for the movement mechanism unit 2.

The potentiometer 32 detects, on the same axis as an output axis of the actuator for the drive unit 12L, 12R, 13L, 13R, the joint angle according to the subject's lower limb movements by detecting the rotation angle of the above-mentioned output axis.

Moreover, the absolute angle sensor 33 is installed in the lower limb frame 11 and measures the absolute angle of the subject's thigh part relative to a vertical direction. This absolute angle sensor 33 is configured of an acceleration sensor and a gyro sensor and is used for a sensor fusion which is a method for extracting new information by using a plurality of sensor data.

A first-order filter is used for the calculation of the absolute angle of the thigh part in order to remove the influences of translational motions and temperature drifts at the respective sensors. This first-order filter is calculated by assigning and adding a weight to values obtained from the respective sensors.

Where θabs(k) represents the absolute angle of the thigh part relative to the vertical direction, w represents the angular velocity obtained by the gyro sensor, dt represents a sampling period, and a represents the acceleration obtained by an acceleration sensor, θabs(t) is expressed as the following Expression (1).

### Math. 1

θ_{abs}(k)=0.95×(θ_{abs}(k−1)+ωdt)+0.05×α . . . (1)

Furthermore, the torque sensor 34 detects, for example, a current value supplied to the drive unit 12L, 12R, 13L, 13R and detects the driving torque by multiplying this current value by a torque constant which is specific to the actuator.

A biosignal detection unit 41 having a bioelectrical potential sensor (electrode group) is located on the subject's body surface region (mainly the body surface of the thigh part) based on the relevant joint in conjunction with the subject's lower limb movements and detects changes in an ionic current transmitted from the subject's cranial nervous system to their muscular system as the biopotential signal which appears on the skin surface.

The biosignal detection unit 41: is a detection unit that measures a nerve action potential generated from the brain towards the legs to move the subject's legs and a muscle action potential generated when the skeletal muscles generate the muscle strength; and has electrodes for detecting faint electric potential at the peripherals of the body system. Incidentally, in this embodiment, the bioelectrical potential sensor is attached to the subject's skin surface with, for example, an adhesive seal to cover around the electrodes in a freely attachable/detachable manner.

The data storage unit 31 stores necessary data for the controller 30 to perform various kinds of arithmetic processing. The biopotential signal detected by the biosignal detection unit 41 is stored in the data storage unit 31. The joint angles (θknee, θhip) detected by the absolute angle sensor 33 of the periarticular detection unit 40 and load data detected by the FRF sensor 60 described later are input to the data storage unit 31.

The controller 30 is configured of, for example, a CPU (Central Processing Unit) chip having a memory and includes a voluntary control unit 50, an autonomous control unit 51, and a synthetic control unit 52.

The voluntary control unit 50 controls the drive units 12L, 12R, 13L, 13R so that a kinetic phenomenon in which the subject's intention to move is reflected will occur based on the biopotential signal and the periarticular physical quantities. Specifically, the voluntary control unit 50 supplies a command signal according to the detection signal of the biosignal detection unit 41 to the electric current control unit 55.

The voluntary control unit 50 generates the command signal by applying a specified command function f(t) or gain P to the biosignal detection unit 41. This gain P is a preset value or function and can be adjusted by external input.

Data of the knee joint angle detected by the potentiometer 32, data of the thigh part absolute angle relative to the vertical direction which is detected by the absolute angle sensor 33, the driving torque detected by the torque sensor 34, and the biopotential signal detected by the biosignal detection unit 41 are input to the data storage unit 31.

Moreover, the foot soles of the pair of dedicated shoes 26L, 26R are provided with the FRF (Floor Reaction Force) sensor 60 to detect a pressure distribution to the subject's right and left foot sole surfaces. This FRF sensor 60 can measure the load imposed on the foot sole surface by dividing the load into a forefoot part (toe part) and a rear foot part (heel part) and measuring the load there independently.

This FRF sensor 60 is composed of, for example, a piezoelectric element(s) for outputting a voltage according to the applied load or a sensor which changes the electrostatic capacity depending on the load, so that the FRF sensor 60 can detect load changes caused by movements of the body weight and whether the subject's leg(s) has touched the floor/ground or not.

Furthermore, with the pair of dedicated shoes 26L, 26R, it is possible to find the gravity center position from the load balance concerning the right and left foot sole surfaces based on the detection results of each FRF sensor 60. Accordingly, with the pair of dedicated shoes 26L, 26R, it is possible to estimate on which side of the subject's right and left feet the center of gravity is biased, based on the data measured by each FRF sensor 60.

Each dedicated shoe 26L, 26R has, other than the shoe structure, the FRF sensor 60, an FRF control unit 61 which is composed of an MCU (Micro Control Unit), and a transmission unit 62. An output from the FRF sensor 60 is transduced to a voltage by a transducer 63 and is then input, with its high frequency band blocked via an LPF (Low Pass Filter) 64, to the FRF control unit 61.

This FRF control unit (a gravity center position detection unit and a walking state recognition unit) 61 finds, based on the detection results of the FRF sensor 60, load changes caused by the subject's body weight shifts and whether the foot soles have touched the floor/ground or not, and also finds the gravity center position according to the load balance relating to the right and left foot soles. The FRF control unit 61 wirelessly transmits the found gravity center position as FRF data, via the transmission unit 62, to a reception unit 65 inside the device.

After the controller 30 receives the FRF data which has been wirelessly transmitted from the transmission unit 62 for each dedicated shoe 26L, 26R via the reception unit 65, the load and the gravity center position relating to the right and left foot soles based on the FRF data are stored in the data storage unit 31.

The autonomous control unit 51: stores respective reference parameters of phases, which are a series of minimum movement units constituting the subject's movement patterns classified as tasks, in the data storage unit 31; estimates the phase of the subject's task by comparing the periarticular physical quantities with the reference parameters stored in the data storage unit 31; and controls the drive unit to generate motive power according to the relevant phase.

The autonomous control unit 51 compares the data of the knee joint angle detected by the periarticular detection unit (the potentiometer 32) 40 and the data of the load detected by the FRF sensor 60 with the knee joint angle and the load which are the reference parameters stored in the data storage unit 31 and estimates the phase of the subject's movement based on the comparison result.

Then, after obtaining control data of the estimated phase, the autonomous control unit 51 generates a command signal according to the control data of this phase and supplies the command signal to the electric current control unit 55 to cause the drive units 12L, 12R, 13L, 13R to generate this motive power.

Moreover, the autonomous control unit 51, to which the gain adjusted by external input is input, generates a command signal according to this gain and outputs the command signal to the electric current control unit 55. The electric current control unit 55 applies an assist force by the actuators to the subject's knee joints by controlling the electric current for driving the actuators for the drive units 12L, 12R, 13L, 13R and thereby controlling a torque size and a rotation angle of the actuator.

Accordingly, the autonomous control unit 51 identifies each walking phase according to the subject's walking task based on the physical quantities detected by the periarticular detection unit (the potentiometer 32, the absolute angle sensor 33, and the torque sensor 34) 40 and causes the drive units 12L, 12R, 13L, 13R to generate the motive power corresponding to each walking phase.

The synthetic control unit 52 synthesizes the control signals from the voluntary control unit 50 and the autonomous control unit 51, causes the electric current control unit 55 to amplify a driving current according to the synthesized control signal, and supplies the amplified driving current to the actuators for the drive units 12L, 12R, 13L, 13R. This torque of the actuators is transmitted, as the assist force, via to the lower limb frames to the subject's knee joints.

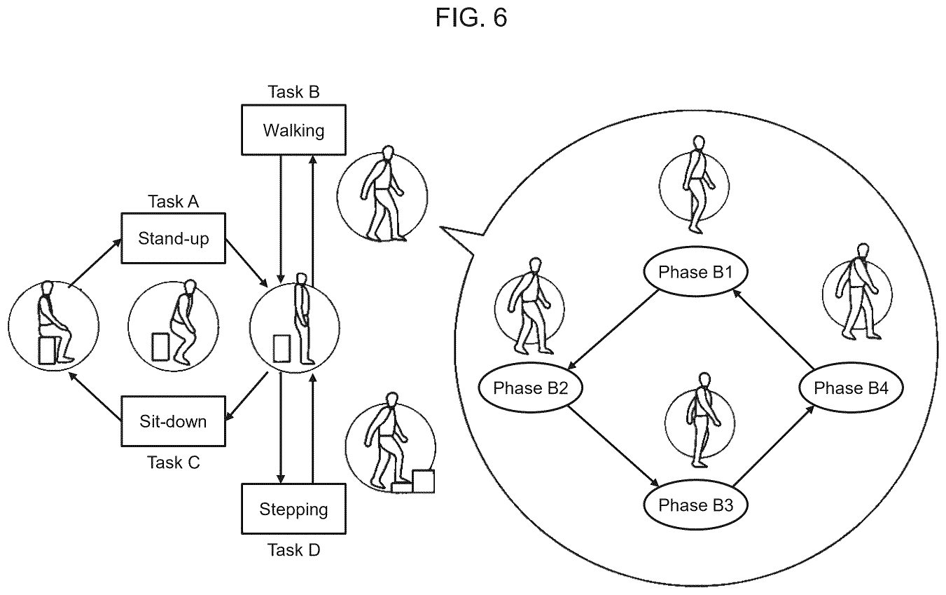

FIG. 6 illustrates an example of the respective tasks and the respective phases which are stored in the data storage unit 31. Regarding the tasks for classifying the subject's movements, for example, the following tasks are stored in the data storage unit 31: Task A having stand-up movement data to make a transition from a seated position to a standing position; Task B having walking movement data for the subject who has stood up to walk; Task C having sit-down movement data to make a transition from the standing position to the seated position; and Task D having stepping movement data to go up and down stairs from the standing position.

Then, a plurality of pieces of phase data are set to each task. For example, the following phases are set to Task B of the walking movement: Phase B having movement data (such as trajectories of the joint angles and the gravity center position, fluctuations of the torque, and changes of the biopotential signal) when the center of gravity is placed on the left leg and the subject intends to swing the right leg forward from a stance leg position; Phase B having movement data from the state with the right leg put forward to touch the floor and shift the center of gravity; Phase B having movement data from the state where the center of gravity is placed on the right leg and the subject tends to swing the left leg forward from the stance leg position; and Phase B having movement data from the state with the left leg put in front of the right leg to touch the floor and shift the center of gravity.

Accordingly, if humans' general movements are analyzed, you can see that a typical movement pattern in each phase such as the angle of each joint, the shifts in the center of gravity, and so on is determined. So, regarding the respective phases constituting humans' many basic movements (tasks), typical positional changes of the joint angles, the status of shifting the center of gravity, and so on are found empirically and these information are stored in the data storage unit 31. Moreover, assist patterns for a plurality of patterns are assigned to each phase, so that even in the same phase, different assists are provided depending on the respective assist patterns.

In the above-described configuration, the movement function improvement device 1 causes the biosignal detection unit 41 to detect changes in the ionic current transmitted from the subject's cranial nervous system to their muscular system as the biopotential signal and operates to apply the driving force from the drive units (actuators) based on this detection signal.

The driving torque according to the biopotential signal generated when the subject acts on their own intention and tries to perform the walking movements is applied as the assist force to the subject wearing the movement mechanism unit 2. Specifically speaking, the assist force is a force to generate a torque which acts on each joint (corresponding to each of the subject's knee joints and hip joints) in the frame mechanism of the movement mechanism unit 2 as a rotation axis.

Therefore, the subject can walk with a total force of their own muscle strength and the driving torque from the drive units 12L, 12R, 13L, 13R, while supporting their body weight. Under this circumstance, the movement function improvement device 1 controls the assist force, which is applied according to the shifts in the center of gravity caused by the walking movements, to reflect the subject's intention. Accordingly, the drive units 12L, 12R, 13L, 13R for the movement mechanism unit 2 are controlled to not impose a load against the subject's intention and is controlled to impede the subject's movements.

Moreover, the movement function improvement device 1 can assist, other than the walking movements, movements corresponding to the subject's intention, for example, the movement of the subject to stand up from the position seated on a chair, or the movement to sit down on a chair from the standing position, and furthermore the movements to go up and down stairs. Particularly, when the muscle strength is weak, it is difficult to perform the movement to go up the stairs or the movement to stand up from the seated position; however, the subject who is wearing the movement mechanism unit 2 can perform the movement(s) with the driving torque applied according to their own intention and without worrying about the degradation of the muscle strength.

The synthetic control unit 52 causes the data storage unit 31 to store the control ratio of the voluntary control unit 50 and the autonomous control unit 51, which is set for each phase of each task, and synthesizes the control states by the voluntary control unit 50 and the autonomous control unit 51 so as to achieve the control ratio according to the relevant phase.

Specifically speaking, when the subject intends to move their body, their intention to move is transmitted as a feeble ionic current from the brain to the spinal cord, the nerves, the muscle spindles, and the muscles and then the musculoskeletal system having the joints will move. When this happens and if the feeble biopotential signal is detected from the subject's skin surface, the voluntary control unit 50 controls the actuators to cause the joints to move according to the subject's intention.

Then, the movement mechanism unit 2 is fastened to, for example, the subject's legs (body parts) in a firmly attached state, so the driving forces of the drive units 12L, 12R, 13L, 13R are transmitted to the subject as assist forces to rotate the joints. Accordingly, the subject's body is moved by the assist force of the movement mechanism unit 2, so that a signal of la afferent neurons return from the muscle spindles through the nerves and the spinal cord to the brain.

Consequently, an interactive biofeedback which is composed of a signal transmission system of “the brain—> the spinal cord—> the motor nerves—> [the musculoskeletal system+the movement mechanism unit 2]” and a signal transmission system of “the movement mechanism unit 2—> the musculoskeletal system (the muscle spindles) —> the sensory nerves—> the spinal cord—> the brain” is configured between the subject, the brain, and the movement function improvement device 1. This is bidirectional voluntary control from the brain and from the movement mechanism unit 2 and the effect of the functional recovery training of the nervous system by neuro-rehabilitation can be further enhanced.

Accordingly, the movement function improvement device 1 is configured to sense the biopotential signal from the brain to the peripherals after the intention/decision regarding movements is made, and to make use of the sensed biopotential signal to control the actuators; and it is designed to recognize the biopotential signal corresponding to the brain activities in terms of activities of the muscles which are the peripherals. Then, it becomes possible to implement real-time feedback of what is sensed at the peripherals to the brain and the nervous system. Consequently, the functional recovery via the directional signal transmission system can be promoted by performing the rehabilitation in the state where the subject wears the movement mechanism unit 2.

Moreover, in a case of severe motor function impairment, the voluntary control does not function in a state where the biopotential signal cannot be detected; and, therefore, the control ratio is switched so that the autonomous control which controls the drive units by using a control program for each phase can function on the basis of the humans' basic movement patterns and the analysis results of the movement mechanism.

By the above-described hybrid control method for which the voluntary control and the autonomous control coexist, the amplitude, signal properties, etc. of the biopotential signal also change according to the status of the bodily motor functions, for example, even in a state where the body is completely paralyzed or in a progression stage of neurological muscular intractable diseases, the rehabilitation can be performed effectively also for these symptoms.

Furthermore, the synthetic control unit 52 compensates for the physical impedance of the entire system, which is composed of the entire device and the subject, based on the periarticular physical quantities in accordance with the physical properties and the gravity of the entire system including the subject's human body properties.

Specifically speaking, the synthetic control unit 52 is configured to form a target motion equation in an arithmetic environment by using motion equation data (Mi) and known parameters (Pk) which are read from the data storage unit 31, and to be capable of assigning an estimated driving torque value (Te), an estimated joint torque value (ΔT), and a joint angle θ to that motion equation.

Under this circumstance, the motion equation data (Mi) are to constitute the motion equation of the entire system which is composed of the movement function improvement device 1 and the subject, while the known parameters (Pk) are formed of kinetic parameters such as the weight of each part of the movement function improvement device 1, a periarticular inertia moment, a viscosity coefficient, and a Coulomb friction coefficient.

The periarticular detection unit 40 has not only the potentiometer 32, the absolute angle sensor 33, and the torque sensor 34 which are described earlier, but also a relative force detection unit 70, an articular torque estimation unit 71, and a muscular torque estimation unit 72. The relative force detection unit 70 detects a relative force (ΔF) which acts on the movement mechanism unit (frame mechanism) 2, that is, a force relatively determined by the relationship between the force generated by the drive units 12L, 12R, 13L, 13R and the subject's muscle strength.

The articular torque estimation unit 71 estimates each periarticular joint moment (ΔT) of the subject from the difference between the result of multiplication of the relative force data (ΔF), which is detected by the relative force detection unit 70, by a preset coefficient and the driving torque (Te) detected by the torque sensor 34. The total force of the driving torque (Te) of the drive units 12L, 12R, 13L, 13R and the subject's muscular torque (Tm) acts as the joint moment (ΔT) on the subject's legs, so the subject can move their legs with smaller muscle strength than the case where the subject is not wearing the movement mechanism unit (frame mechanism) 2.

The muscular torque estimation unit 72 estimates the muscular torque (Tm) by the subject's muscle strength based on the driving torque (Te) detected by the torque sensor 34 and the joint moment (ΔT) estimated by the articular torque estimation unit 71. Incidentally, the muscular torque (Tm) is obtained in order to enable the parameter identification even under the circumstance where the subject generates the muscle strength; and it is advantageous for the case of performing the parameter identification when the subject is in a moving state.

The synthetic control unit 52: performs arithmetic processing in consideration of the driving torque (Te), the joint data (θ) and the joint moment (ΔT) which are obtained from the periarticular detection unit 40, and also the muscular torque (Tm); identifies unknown kinetic parameters (Pu) such as the weight of each part of the subject, each periarticular inertia moment, the viscosity coefficient, and the Coulomb friction coefficient, and averages the obtained data by repeating the above-described processing multiple times (for example, 10 times).

Subsequently, the synthetic control unit 52 reads a ratio of the estimated muscular torque (Tm) to the bioelectrical potential (Tm/BES), and a specified set gain (Gs) from the data storage unit 31; and if the set gain (Gs) is out of an allowable error range (Ea), the synthetic control unit 52 corrects the bioelectrical potential (BES) to obtain a corrected bioelectrical potential (BES′) and makes a ratio of the muscular torque (Tm) to the corrected bioelectrical potential (BES′) (Tm/BES′) substantially equal to the set gain (Gs).

As a result, it is possible to prevent the situation where the identification accuracy of the subject's unknown kinetic parameters (Pu) may degrade; and it is also possible to prevent the situation where the assist forces generated by the drive units 12L, 12R, 13L, 13R may become too small or too large.

The synthetic control unit 52 is configured to be capable of reading the control method data (Ci) from the data storage unit 31, the driving torque (Te), the joint torque (ΔT) and the joint angle θ which are obtained from the periarticular detection unit 40, as well as the identified parameters (Pi) which are the identification results of the unknown kinetic parameters (Pu), and the corrected bioelectrical potential (BES′).

Moreover, the synthetic control unit 52 configures a specified control unit in the arithmetic environment by using the control method data (Ci) and causes the driving torque (Te), the joint torque (66 T), the joint angle θ, the identified parameters (Pi), and the bioelectrical potential (BES′) to be reflected in this synthetic control unit 52, so that it can transmit a control signal Ur for performing drive control of the drive units 12L, 12R, 13L, 13R. The electric current control unit 55 drives the drive units 12L, 12R, 13L, 13R according to the control signal Ur from the synthetic control unit 52.

Furthermore, the movement function improvement device 1 is designed to control the assist forces based on the impedance adjustments in order to solve natural control impedance attributable to restraints by physical properties of the device itself, that is, the periarticular viscoelasticity and the inertia of the frames. Specifically speaking, the movement function improvement device 1 calculates joint parameters and causes the drive units (the actuators) 12L, 12R, 13L, 13R to compensate for the inertia moment, viscosity, and elasticity, thereby improving an assist rate in the walking movements and alleviating the subject's discomfort.

Accordingly, the movement function improvement device 1 can indirectly change and adjust the subject's characteristics by changing the properties of the entire system, that is, the device itself and the subject in addition thereto. For example, it becomes possible for the subject to exert their ability to make agile movements, such as reflexes which the subject inherently has, to the maximum by adjusting the driving torque so that the influences of an inertia term and a viscous friction term of the entire system can be suppressed. Furthermore, it is possible to suppress even the influences of the subject's own inertia term and viscous friction term and it is also possible to make the subject walk in faster cycles than their inherent cycles or move more smoothly (with less viscous frictions) than before wearing the device.

Furthermore, the movement function improvement device 1 can cause the synthetic control unit 52 to identify the subject's inherent kinetic parameters in the state of being mounted on the subject and cause the controller 30 to control the drive units 12L, 12R, 13L, 13R based on the motion equation to which the identified kinetic parameters are applied, so that it is possible to exert the effects according to the control method used by the controller 30 regardless of fluctuating causes of the subject's individual differences and health condition.

Moreover, the drive units 12L, 12R, 13L, 13R can be controlled by the controller 30 based on the motion equation to which the muscular torque (Tm) estimated by the periarticular detection unit 40 is applied, so that even in the state where the muscle strength is generated from the subject, the kinetic parameters can be identified and the above-described effects can be exerted without requiring waiting time for the subject to wait for the identification of the kinetic parameters.

Since a mutual gain between the bioelectrical potential (BES) detected by the biosignal detection unit 41 and the muscular torque (Tm) detected by the periarticular detection unit 40 is adjusted to become the preset set gain (Gs), it is possible to prevent the situation in advance where any poor sensitivity or excessive sensitivity may occur as the detection result from the biosignal detection unit 41.

As a result, it is possible to prevent the situation where the identification accuracy of the subject's kinetic parameters may degrade; and it is also possible to prevent the situation where the assist forces generated by the drive units 12L, 12R, 13L, 13R may become too small or too large. Moreover, even in the state where the muscle strength is generated from the subject, the movement function improvement device 1 in this embodiment can perform calibration and does not require the waiting time for the subject to wait for the calibration.

Since at least either gravity compensation or inertia compensation using the kinetic parameters identified by the synthetic control unit 52 can be applied to the controller 30, it is possible to prevent the situation where the weight of the device itself may give a burden to the subject or the situation where the inertia of the device itself during movements may make the subject feel discomfort.

Additionally, in order to minimize the difference between the subject's intention to move and the kinetic phenomenon, the synthetic control unit 52 corrects the above-described difference by the movement command from the cranial nervous system according to the biofeedback loops interactively promoted between the subject's body and the movement mechanism unit 2 and, at the same time, feedback-adjusts the synthesized control state.

As a result, with the movement function improvement device 1, when the subject repeats performing the voluntary movements by using the movement mechanism unit 2, the motor functions of the subject's brain, nerves, and muscular system can be improved in conjunction with the correction of the difference between the movement command from the cranial nervous system and the actual kinetic phenomenon.

Furthermore, with the movement function improvement device 1, when forming the biofeedback loops, the minimum motion control unit(s) for realizing the voluntary movements generated by the subject's intention is set as the minimum voluntary motor control unit(s) composed of the cranial nervous system, the synaptic connections, and the muscular system and the biofeedback loops are established for each minimum voluntary motor control unit which forms the body movements which work cooperatively in order to realize the kinetic phenomenon by using the movement mechanism unit 2.

As a result, with the movement function improvement device 1, by explicitly incorporating the minimum voluntary motor control unit, in which the influences of the diseased area and the disease cause are reflected, into the process of the functional improvement and treatment based on the basic theory of the biofeedback loops by the movement mechanism unit, other minimum voluntary motor control units are also influenced and synchronize with the movements of the movement mechanism unit 2, which causes their cooperative movements to progress; and for the purpose of realization of the aimed movement, the functions of the respective minimum voluntary motor control units synchronize with the movements of the movement mechanism unit 2 and are enhanced and adjusted, thereby making it possible to achieve the functional improvement of the brain, the nerves, and the muscular system.

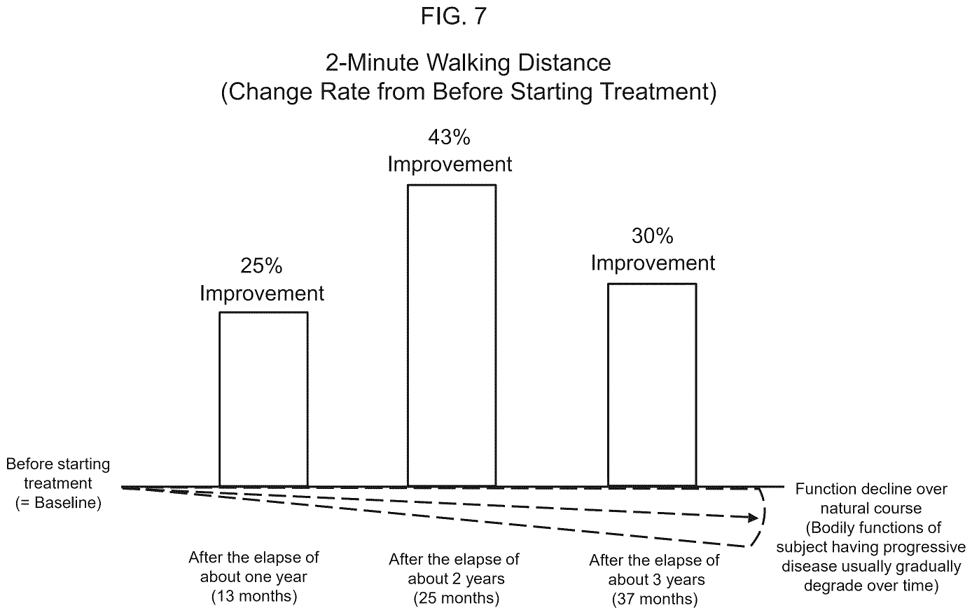

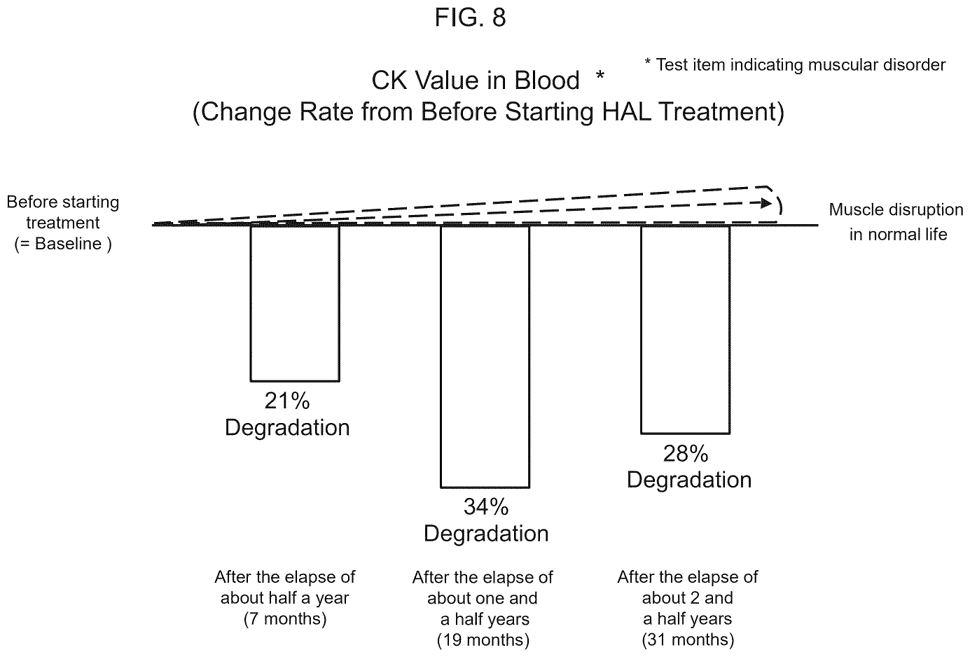

For example, regarding the subject who has a progressive disease (slowly progressive neuromuscular disease), their movement functions usually gradually degrade over time; however, when the movement function improvement device 1 is used, the effect of functional improvements which are indicated in FIG. 7 and could not be expected conventionally can be obtained. In normal life and in conventional exercise therapies, it is considered common sense that muscle disruption causes a CK value in blood, which is an index indicating the muscle disruption in blood, to rise; however, the effect of rather reducing the CK value can be obtained by the movement function improvement device 1 (FIG. 8).

### (4) Movement Function Improvement Device According to Another Embodiment

### (4-1) Configuration of Movement Mechanism Unit (Lower Back Type: Hardware)

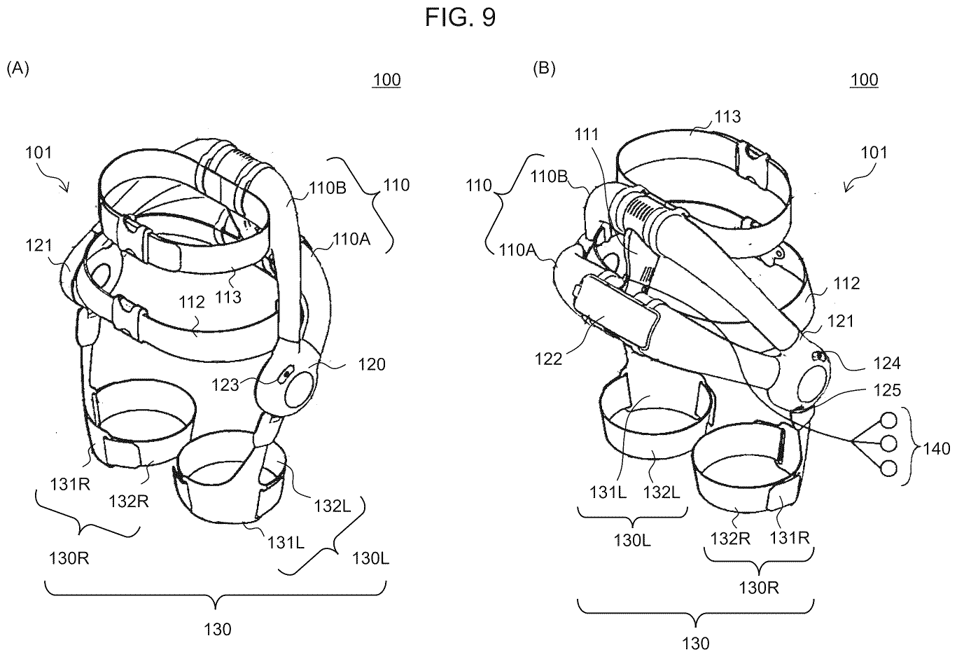

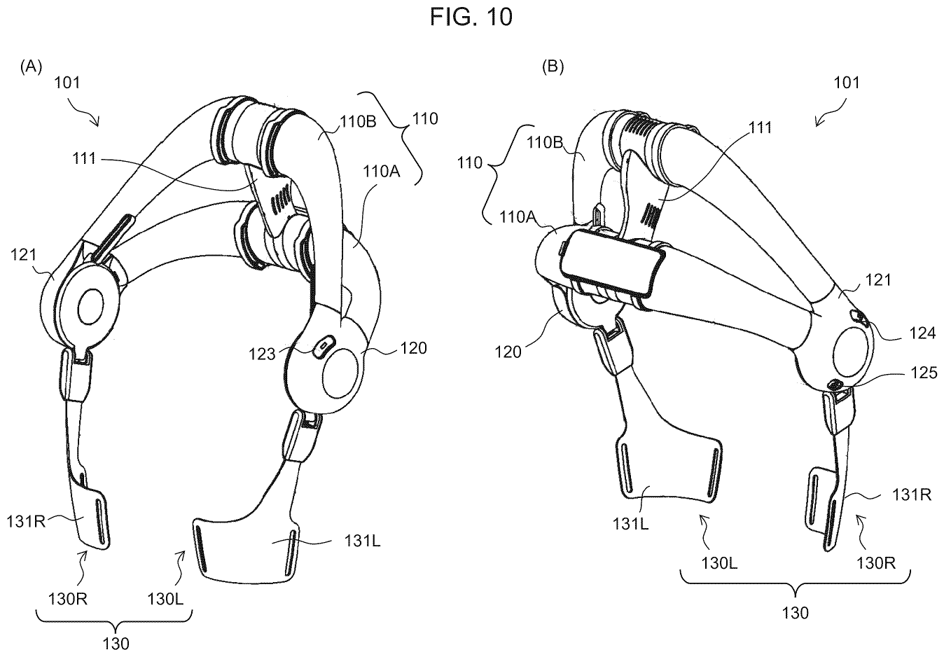

FIGS. 9(A) and 9(B) illustrate a movement function improvement device 100 of a lower back type according to another embodiment. Moreover, FIGS. 10(A) and 10(B) illustrate a major configuration excluding thigh cuffs, belts, and so on of a movement mechanism unit 101 in FIG. 9. The movement function improvement device 100 is a device that supports (assists) the subject's work and movements, detects the biopotential signal, movement angles of the subject's hip joints and an absolute angle of their body trunk, and operates to apply driving forces from the drive units based on this detection signal.

When the subject who is wearing the movement function improvement device 100 performs, with their own intention, movements to lift and carry a relatively heavy object, the biopotential signal which is then generated on skin surfaces of latissimus dorsi or gluteus maximus (gluteus maximus muscle) and driving torques according to the movement angles of the subject's hip joints are applied as assist forces from the movement mechanism unit 101. Therefore, the subject can lift up and carry the object with a total force of their own muscle strength and the driving torques from the drive mechanism (actuator).

Moreover, the movement function improvement device 100 can also assist, other than the carrying work to lift up the object and walk, for example, stepping work for the subject to go up and down stair while holding luggage.

The movement mechanism unit 101 for the movement function improvement device 100 is equipped with a lower-back-part frame 110 which extends to the right and left directions on the back side of the subject's lower back. The lower-back-part frame 110 is, for example, a hollow member made of CFRP (carbon fiber reinforced plastic) and has a rounded shape in conformity to the shapes of the back side and both lateral sides of the human body's lower back part.

The lower-back-part frame 110 is configured so that on the back-face side of the subject's lower back part, a first lower-back-part frame 110A which is mounted to extend to the right and left sides and a second lower-back-part frame 110B which is mounted to extend to the right and left directions above the first lower-back-part frame 110A are connected via a support pole 111.

The first lower-back-part frame 110A and the second lower-back-part frame 110B are mounted on the subject's lower back part with wearing belts 112, 113 which are put around the subject's abdomen side. The first lower-back-part frame 110A and the second lower-back-part frame 110B are mounted in a bent-forward posture so that both end sides become lower than the back-face side in the mounted state, that is, both ends are located at positions lower than the central part in the longitudinal direction (the portion located on the subject's back-face side).

A left-side-part frame 120 and a right-side-part frame 121 are fixed to both ends of the first lower-back-part frame 110A and the second lower-back-part frame 110B.

Moreover, a battery 122 is housed in the central part of the first lower-back-part frame 110A in a freely attachable/detachable manner on the outside of the movement mechanism unit 101, that is, on the opposite side of its attachment side, and wiring or the like to be connected to the battery 122 is inserted through its inside space.

The support pole 111 is a member for connecting, in a vertical direction, a lengthwise-direction central part of the first lower-back-part frame 110A and a lengthwise-direction central part of the second lower-back-part frame 110B. The support pole 111 is, for example, a hollow member made of reinforced resin and wiring for sensors is inserted into the support pole 111. Moreover, a controller (which is not illustrated in the drawing, but corresponds to the controller 30 in FIG. 5 described earlier) for controlling movements of the movement mechanism unit 101 is provided inside the support pole 111.

As various kinds of members which configure the first lower-back-part frame 110A and the second lower-back-part frame 110B are retained by the support pole 111 so that they will not rotate, the strength of a monocoque structure is ensured. Moreover, the wearing belt 112 corresponding to the first lower-back-part frame 110A and the wearing belt 113 corresponding to the second lower-back-part frame 110B are respectively attached to the support pole 111.

The left-side-part frame 120 joins the left end of the first lower-back-part frame 110A and the left end of the second lower-back-part frame 110B together and secure them on the left side of the subject's hip joint. The right-side-part frame 121 has the configuration which is substantially bilaterally symmetrical to that of the left-side-part frame 120, and joins the right end of the first lower-back-part frame 110A and the right end of the second lower-back-part frame 110B together and secure them.

An actuator and a brake mechanism (both of which are not illustrated in the drawing) are built in the left-side-part frame 120, which is provided with a minus button 123 for input to reduce a driving force of the actuator. This minus button 123 lights up when the movement function improvement device is in a power-on state.

An actuator and a brake mechanism (both of which are not illustrated in the drawing) are built in the right-side-part frame 121, which is provided with a plus button 124 for input to increase a driving force of the actuator and a power button 125 to switch power on/off of the movement function improvement device 1. This plus button 124 and the power button 125 light up when the movement function improvement device is in a power-on state.

Accordingly, the lower-back-part frame (the first lower-back-part frame 110A and the second lower-back-part frame 110B), the support pole 111, and the side part frames (the left-side-part frame 120 and the right-side-part frame 121) are assembled to form an integrated configuration, thereby realizing the monocoque structure which causes the frames themselves to be subjected to stresses.

In FIGS. 9(A) and 9(B), a thigh securing part 130 is composed of a left thigh securing part 130L for securing the subject's left-side thigh part and a right thigh securing part 130R for securing the subject's right-side thigh part.

The left thigh securing part 130L is configured from a stay part 131L connected to the actuator inside the left-side-part frame 120 and a belt part 132L attached to the stay part 131L and is provided to be rotatable relative to the left-side-part frame 120 as viewed from the lateral side.

Moreover, the right thigh securing part 130R is configured from a stay part 131R connected to the actuator inside the right-side-part frame 121 and a belt part 132R attached to the stay part 131R and is provided to be rotatable relative to the right-side-part frame 120 as viewed from the lateral side.

Incidentally, regarding the left thigh securing part 130L and the right thigh securing part 130R, the stay part 131L, 131R is designed to have an optimum length with reference to an average length of a thigh part of a human body and the subject's thigh part is secured by the belt part 132L, 132R.

The wearing belt 112 is a wearing blet which is put around the abdomen side when attaching the first lower-back-part frame 110A to the subject and is a main part used to attach the movement mechanism unit 101 to the lower back part of the human body.

The wearing belt 113 is a wearing belt which is put around the abdomen side when attaching the second lower-back-part frame 110B to the subject. The wearing belt 113 is used to secure the movement mechanism unit 101 to the human body at a position above the wearing belt 113 in order to efficiently transmit a reaction force generated along with the leg movements to the subject's abdomen or lower back part when the subject who is wearing the movement mechanism unit 101 performs the movements to lift up the relatively heavy object from a knee-bent position.

The battery 122 is provided on the outside of the central part of the first lower-back-part frame 110A on the outside of the movement mechanism unit 101, that is, on the opposite side of its attachment side and supplies electric power to the controller, the actuators, the brake mechanism (which is not illustrated in the drawing), the minus button 123, the plus button 124 and the power button 125.

A biosignal detection unit 140 having bioelectrical potential sensors is a detection unit which is mounted on the back side of the subject's lower back part and detects a biopotential signal caused in conjunction with muscle activities when the subject tries to raise their body trunk or muscle activities when the subject tries to keep the angle of the body trunk.

The bioelectrical potential sensors for the biosignal detection unit 140 are connected to a top end of wiring extending outside of the support pole from a hole made in the support pole 111 and there are three bioelectrical potential sensors. The bioelectrical potential sensors are pasted on the back side of the subject's lower back part, thereby sensing the biopotential signal generated when the subject moves the muscles of their body trunk.

The biopotential signal detected by the biosignal detection unit 140 is input to the controller. Incidentally, the bioelectrical potential sensor may be pasted on the subject's back by using a tape or the like or may be pasted by using a gel or the like. One of the three sensors is used to measure a reference signal and the remaining two sensors are used to measure the biopotential signal.

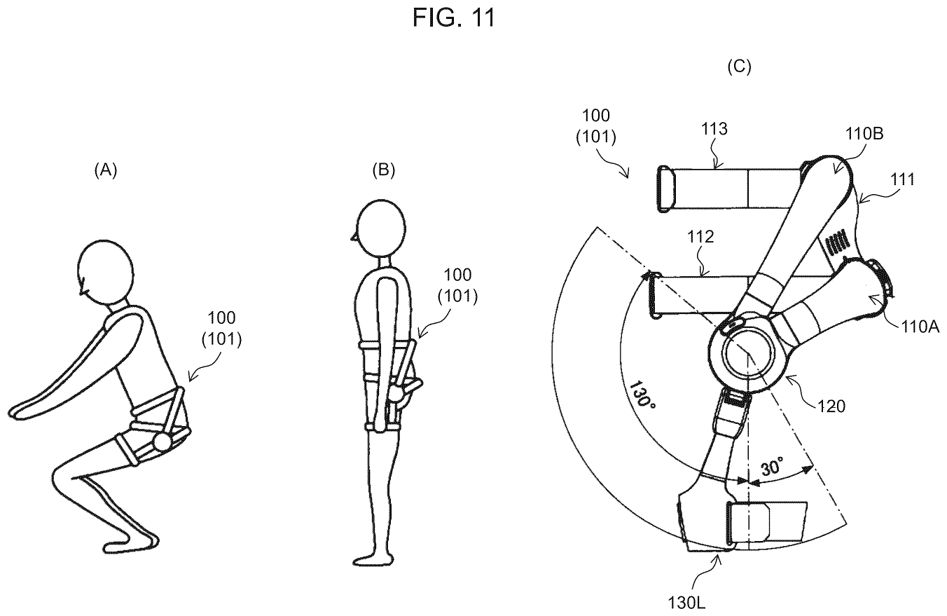

Practically, regarding the movement function improvement device 100, the movement mechanism unit 101 is mounted on the subject's lower back part from the back-face side. The movement function improvement device 100 is a device which generates an assisting force (assist force) to assist the movements of the thigh parts relative to the lower back part when the subject stands up as illustrated in FIG. 11(B) from a semi-crouching state (half-sitting posture) as illustrated in FIG. 11(A). The above-described movements of the subject are, for example, movements to stand up from the half-sitting posture, movements to lift up an object from the half-sitting posture, and movements for transfer assistance.

FIG. 11(C) is a diagram (a left side view) illustrating a movable range of the movement mechanism unit 101 for the movement function improvement device 100. The left thigh securing part 130L can rotate by 130° clockwise and by 30° counterclockwise from a reference position indicated in FIG. 11(C) around the left-side-part frame 120 as a rotation center. With such movements cause the movement function improvement device to generate the assisting force to assist the movements of the thigh parts relative to the lower back part when the subject stands up as illustrated in FIG. 11(B) from the semi-crouching state as illustrated in FIG. 11(A). Incidentally, the movement range of the right thigh securing part 130R is similar to that described above.

The movement function improvement device 100 of the lower back type in this embodiment has almost the same configuration (excluding the reception unit 65) of that of the control system 3 for the movement function improvement device 1 of the aforementioned lower limb type. Accordingly, the movement function improvement device 100 also functionally includes: a voluntary control step of causing movements according to the subject's intention to move; an autonomous control step of causing preset ideal motive power; and an impedance control step (including gravity compensation control) of alleviating a sense of difficulty to move due to the load and viscous frictions of the movement mechanism unit 101 itself.

As a result, with the movement function improvement device 100 of the lower back type, the subject can feel as if the movement mechanism unit 101 were part of their own body, so it is possible to realize functional fusion and integration between the subject and the movement mechanism unit 101.

Practically, with the movement function improvement device 100, it is possible to generate the assisting force to assist the movements of the thigh parts relative to the lower back part when the subject stands up as illustrated in FIG. 11(B) from the semi-crouching state as illustrated in FIG. 11(A). Such assisting force is generated as a result of driving the drive units (actuators) based on the biopotential signal which is detected by the biosignal detection unit 140 and which is caused in conjunction with the muscle activities when the subject tries to raise their body trunk or the muscle activities when the subject tries to keep the angle of their body trunk.

Therefore, it is possible to provide the movement function improvement device 100 which is capable of providing the necessary assisting force according to the subject's intention in a necessary direction and which is highly convenient. It is also possible to provide the movement function improvement device 100 which can suppress the motive power (muscle strength) that should be generated by the subject themselves as much as possible and which can prevent the situation where the subject's convenience may be impaired.

Incidentally, if the controller determines that the biosignal signal levels of the subject's right and left thighs are not equally balanced, it judges that the subject is walking; and if the controller determines that the biosignal signal levels of the subject's right and left thighs are equally balanced, it judges that the subject is in a stopped state.

Moreover, in the state where the controller judges that the subject is in the stopped state, if it determines that both right and left hip joint angles are larger than a predetermined specified angle, it judges that the subject is walking; and on the other hand, if it determines that both the right and left hip joint angles are equal to or smaller than the specified angle, it judges that the subject is in a posture with their upper body lowered forwardly.

Furthermore, with the movement function improvement device 100, the synthetic control step of synthesizing the control states by the voluntary control step and the autonomous control step in order to obtain the control ratio according the phase of the task not only executes the aforementioned impedance control step, but also corrects the difference between the subject's intention to move and the kinetic phenomenon by the movement command from the cranial nervous system and, at the same time, feedback-adjusts the synthesized control state on the basis of the biofeedback loops promoted interactively between the subject's body and the movement mechanism unit in order to minimize the above-described difference.

As a result, when the subject repeatedly performs the voluntary movements by using the movement mechanism unit 101, the movement function improvement device 100 can improve the motor functions of the subject's brain, nerves, and muscular system in conjunction with the correction of the difference between the movement command from the cranial nervous system and the actual kinetic phenomenon.

Furthermore, with the movement function improvement device 100, when forming the biofeedback loops, the minimum motion control unit(s) for realizing the voluntary movements according to the subject's intention is set as the minimum voluntary motor control unit(s) composed of the cranial nervous system, the synaptic connections, and the muscular system; and the biofeedback loops are established for each minimum voluntary motor control unit which forms the body movements that work cooperatively to realize the kinetic phenomenon by using the movement mechanism unit 101.

As a result, with the movement function improvement device 100, by explicitly incorporating the minimum voluntary motor control unit, in which the influences of the diseased area and the disease cause are reflected, into the process of the functional improvement and treatment based on the basic theory of the biofeedback loops by the movement mechanism unit, other minimum voluntary motor control units are also influenced and synchronize with the movements of the movement mechanism unit 101, which causes their cooperative movements to progress; and for the purpose of realization of the aimed movement, the functions of the respective minimum voluntary motor control units synchronize with the movements of the movement mechanism unit and are enhanced and adjusted, thereby making it possible to achieve the functional improvement of the brain, the nerves, and the muscular system.

### (3-2) Configuration of Movement Mechanism Unit (Single Joint Type: Hardware)

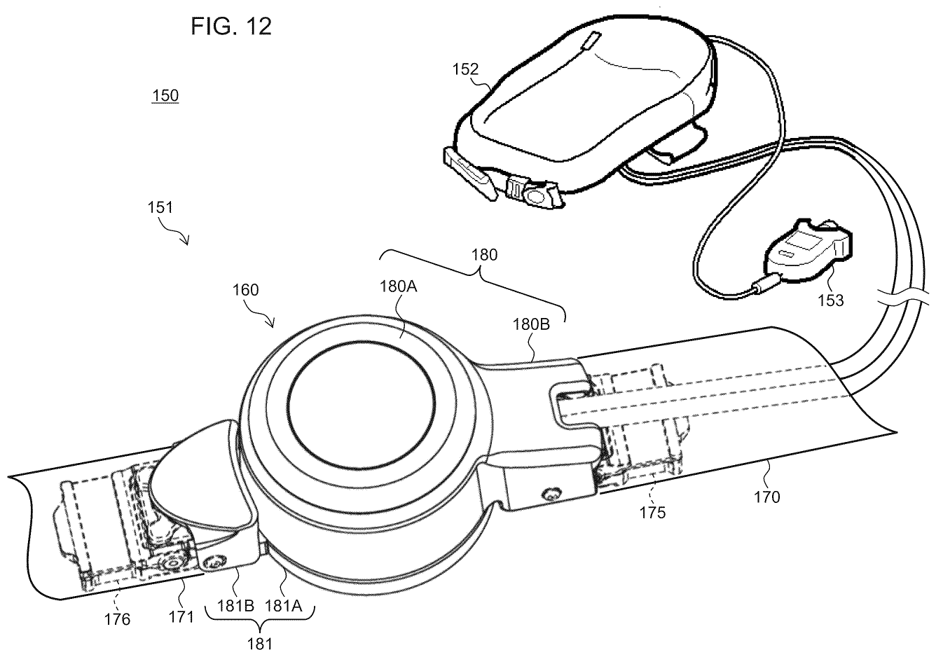

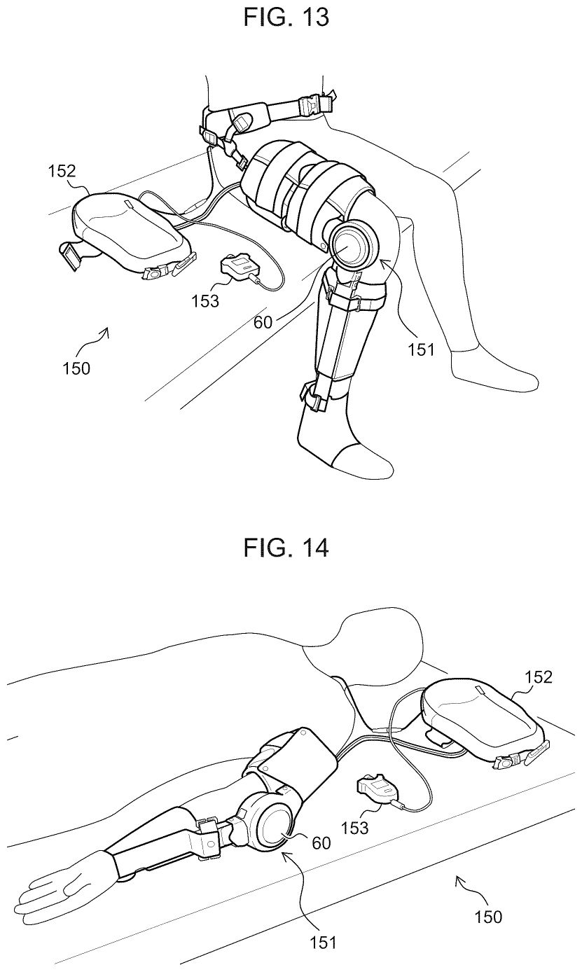

FIG. 12 illustrates a movement function improvement device 150 including a movement mechanism unit 151 of a single joint type according to another embodiment. A major configuration, excluding cuffs, belts, etc., of the movement mechanism unit 151 for this movement function improvement device 150 is illustrated.

The movement function improvement device 150 is a device which is mounted on a joint site of the subject's upper limb or lower limb (such as an elbow joint or a knee joint) to support (assist) the movements of the movement mechanism unit 151; and has a function that improves smoothness of movements of the upper limb or the lower limb by driving a drive unit (actuator) according to the subject's intention to move to assist the voluntary movements and performing repetitive motions of joint extension movements.

The movement function improvement device 150 according to this embodiment is configured, as illustrated in FIG. 12, from the movement mechanism unit 151 which is mounted at an arbitrary joint site of the subject, a control unit 152 in which a controller (which is not illustrated in the drawing, but corresponds to the controller 30 in FIG. 5 described earlier) is built in, and an operation unit 153 for displaying necessary information and performing operations of setting changes and checks. Incidentally, electrode cables and a charger connected to the control unit 152 are omitted in FIG. 12.

The movement mechanism unit 151 is configured to cause a first transmission member 170 and a second transmission member 171 to relatively rotate with a driving force of a drive unit (actuator) 160. These first transmission member 170 and second transmission member 171 are attachments to be selected according to the subject's joint and are fastened to one end-side site and the other end-side site of the relevant joint via a first coupling part 175 and a second coupling part 176.

The drive unit 160 is provided to be positioned on a lateral side of the subject's knee joint and has a first motor housing 180 in which either a rotator (magnet) or a stator (coil) is placed, and a second motor housing 181 in which the other one of the rotator (magnet) and the stator (coil) is placed.

The first motor housing 180 is provided with a first coupling part to which a first transmission member to be mounted on the one end-side site (for example, the thigh part) of the subject's joint is coupled. Moreover, the second motor housing 181 is provided with a second coupling part 38 to which a second transmission member 50 to be mounted on the other end-side site (for example, a shank) of the relevant joint is coupled. Furthermore, the first transmission member 40 has a pair of cuffs (which are not illustrated in the drawing) which engage with the one end-side site of the subject's joint and which are secured thereto via a securing member 76.

The drive unit 160 is configured so that the first motor housing 180 and the second motor housing 181 are placed one over another along the same axis and a back side of the first motor housing 180 and a back side of the second motor housing 181 face opposite each other. Moreover, a rotation center of the first motor housing 180 and a rotation center of the second motor housing 181 are connected together via a motor shaft (which is not illustrated in the drawing) installed between the housings.

Furthermore, the first motor housing 180 has a circular cover 180A formed in a circular dome shape and an extension part 180B formed to extend in a radial direction from the outer circumference of the circular cover 180A. The end of the extension part 180B is formed to support the first coupling part 175 and guide insertion/removal operations of the first transmission member 170. Similarly, the second motor housing 181 has a circular cover 181A formed in a circular dome shape and an extension part 181B formed to extend in a radial direction from the outer circumference of the circular cover 181A. The end of the extension part 181B is formed to support the second coupling part 176 and guide insertion/removal operations of the second transmission member 171.

Regarding the drive unit 160, a relative rotation angle θ between the first motor housing 180 and the second motor housing 181 is set, for example, θ=65 degrees to 180 degrees according to a movement range of the knee joint. Moreover, when the relative rotation angle θ reaches the minimum angle θmin=65 degrees and the maximum angle θmax=180 degrees, a stopper between the first motor housing 180 and the second motor housing 181 comes into contact with them to regulate the relative rotations.

Furthermore, the drive unit 161 has an angle sensor for detecting the relative rotation angle θ between the first motor housing 180 and the second motor housing 181. Incidentally, this angle sensor is housed inside either the first motor housing 180 or the second motor housing 181.

The movement function improvement device 150 of the single joint type according to this embodiment has almost the same configuration (excluding the reception unit) as that of the control system 3 for the movement function improvement device 1 of the aforementioned lower limb type. Accordingly, the movement function improvement device 150 also functionally includes: a voluntary control step of causing movements according to the subject's intention to move; an autonomous control step of causing preset ideal motive power; and an impedance control step (including gravity compensation control) of alleviating a sense of difficulty to move due to the load and viscous frictions of the movement mechanism unit 151 itself.

As a result, with the movement function improvement device 150 of the single joint type, the subject can feel as if the movement mechanism unit 151 were part of their own body, so it is possible to realize functional fusion and integration between the subject and the movement mechanism unit 151.

Practically, with the movement function improvement device 150, by mounting the movement mechanism unit 151 on the knee joint of the subject's right leg as illustrated in FIG. 13, it is possible to perform functional recovery training (rehabilitation) after undergoing treatment or surgery of the subject's right knee joint. Moreover, the functional recovery training can be performed by using the movement function improvement device 150 even when the subject is in a state of lying in bed or when the subject is in a state of being seated on a chair, so it is possible to perform the functional recovery training as appropriate without putting an unreasonable burden on the subject.

Moreover, with the movement function improvement device 150, by mounting the movement mechanism unit 151 on the elbow joint of the subject's left arm, it is possible to perform the functional recovery training (rehabilitation) after undergoing the treatment or surgery of the subject's left elbow joint.

Furthermore, with the movement function improvement device 150, the synthetic control step of synthesizing the control states by the voluntary control step and the autonomous control step in order to obtain the control ratio according the phase of the task not only executes the aforementioned impedance control step, but also corrects the difference between the subject's intention to move and the kinetic phenomenon by the movement command from the cranial nervous system and, at the same time, feedback-adjusts the synthesized control state on the basis of the biofeedback loops promoted interactively between the subject's body and the movement mechanism unit in order to minimize the above-described difference.

As a result, when the subject repeatedly performs the voluntary movements by using the movement mechanism unit 151, the movement function improvement device 150 can improve the motor functions of the subject's brain, nerves, and muscular system in conjunction with the correction of the difference between the movement command from the cranial nervous system and the actual kinetic phenomenon.

Furthermore, with the movement function improvement device 150, when forming the biofeedback loops, the minimum motion control unit(s) for realizing the voluntary movements according to the subject's intention is set as the minimum voluntary motor control unit(s) composed of the cranial nervous system, the synaptic connections, and the muscular system; and the biofeedback loops are established for each minimum voluntary motor control unit which forms the body movements that work cooperatively to realize the kinetic phenomenon by using the movement mechanism unit 151.

As a result, with the movement function improvement device 150, by explicitly incorporating the minimum voluntary motor control unit, in which the influences of the diseased area and the disease cause are reflected, into the process of the functional improvement and treatment based on the basic theory of the biofeedback loops by the movement mechanism unit 151, other minimum voluntary motor control units are also influenced and synchronize with the movements of the movement mechanism unit 151, which causes their cooperative movements to progress; and for the purpose of realization of the aimed movement, the functions of the respective minimum voluntary motor control units synchronize with the movements of the movement mechanism unit 151 and are enhanced and adjusted, thereby making it possible to achieve the functional improvement of the brain, the nerves, and the muscular system.

### (5) Other Embodiment

Incidentally, this embodiment has been described above about the case where the movement function improvement device including the movement mechanism unit 2 for the lower body (the lower limb type), or such device including the movement mechanism unit 101 of the lower back type, or such device further including the movement mechanism unit 151 of the single joint type is applied as the movement function improvement device 1, 100, 150; however, the present invention is not limited to this example and can be widely applied to the movement function improvement device including the movement mechanism unit which is applied to the relevant joint site as long as the joint site is capable of the subject's body movements.

For example, a movement function improvement device including a movement mechanism unit for a hand and fingers (hand type) and a control system equipped with the aforementioned functional configuration of the voluntary control step, the autonomous control step, the impedance control step, and the basic theory of the biofeedback loops may be configured. This movement mechanism unit of the hand type may be configured to be directly attached to the subject's hand and fingers or may be configured to be secured on a table.

Moreover, the aforementioned embodiment has been described about the case where the periarticular detection unit 40 detects, as the periarticular physical quantities, the absolute angle, the rotation angle, the angular velocity, the angular acceleration, and the driving torque between the rotator-side frame and the stator-side frame for the drive units 12L, 12R, 13L, 13R (160) in the movement mechanism unit 2 (101, 151); however, the present invention is not limited to this example and, for example, at least one or more parameter identification results of muscle strength generated in the musculoskeletal, a motion range, a movable speed, and a reaction rate of each joint, autonomous control characteristics against external disturbances, inertia moment, mass, and the center of gravity of the frames, impedance adjustment results (viscous properties due to frictions) of the articular system including flexor muscles and extensor muscles, and electrical physical quantities (command signals) may be detected as the periarticular physical quantities.

### REFERENCE SIGNS LIST

1, 100, 150: movement function improvement device
2, 101, 151: movement mechanism unit
3: control system
12L, 12R, 13L, 13R, 160: drive units
26L, 26R: dedicated shoes
30: controller
31: data storage unit
32: potentiometer
33: absolute angle sensor
34: torque sensor
40: periarticular detection unit
41, 140: biosignal detection unit
50: voluntary control unit
51: autonomous control unit
52: synthetic control unit
55: electric current control unit
60: FRF sensor
70: relative force detection unit
71: articular torque estimation unit
72: muscular torque estimation unit

1. A movement function improvement device comprising:
a movement mechanism unit that is used in a manner united with a subject and has a drive unit to be driven actively or passively in conjunction with body movements of the subject;
a signal detection unit that detects changes of an ionic current transmitted from a cranial nervous system to a muscular system of the subject as a biopotential signal appearing on a skin surface;
a periarticular detection unit that detects periarticular physical quantities caused by body movements of the subject based on an output signal from the drive unit;
a voluntary control unit that controls the drive unit to cause the subject's intention to move to be reflected in a kinetic phenomenon based on the biopotential signal and the periarticular physical quantities;
an autonomous control unit that stores respective reference parameters of phases which are a series of minimum movement units constituting the subject's movement patterns classified as tasks, estimates a phase of the subject's task by comparing the periarticular physical quantities with the reference parameters stored in the data storage unit, and controls the drive unit to generate motive power according to the phase; and
a synthetic control unit that stores a control ratio of the voluntary control unit and the autonomous control unit, which is set for each phase of each task, in the data storage unit and synthesizes control states by the voluntary control unit and the autonomous control unit so as to achieve the control ratio according to the relevant phase,
wherein the synthetic control unit:
compensates for physical impedance of an entire system, which is composed of the entire device and the subject, based on the periarticular physical quantities in accordance with physical properties and gravity of the entire system including human body properties of the subject; and
corrects a difference between the subject's intention to move and the kinetic phenomenon by a movement command from the cranial nervous system and, at the same time, feedback-adjusts the synthesized control state on the basis of biofeedback loops interactively promoted between the subject's body and the movement mechanism unit in order to minimize the difference between the subject's intention to move and the kinetic phenomenon.

2. The movement function improvement device according to claim 1, comprising:
a gravity center position detection unit that detects a gravity center position of the subject based on a load change on foot sole surfaces of the subject; and
a walking state recognition unit that recognizes a walking state of the subject based on the control state by the synthetic control unit and the gravity center position by the gravity center position detection unit,
wherein the voluntary control unit controls the drive unit to cause the subject's intention to move to be reflected in the kinetic phenomenon on the basis of the biopotential signal, the periarticular physical quantities, and the walking state recognized by walking state recognition unit.

3. The movement function improvement device according to claim 1,
wherein when forming the biofeedback loops, a minimum motion control unit for realizing voluntary movements caused by the subject's intention is set as a minimum voluntary motor control unit composed of the cranial nervous system, synaptic connections, and the muscular system; and
wherein the biofeedback loops are established for each minimum voluntary motor control unit that forms body movements which are performed cooperatively to realize the kinetic phenomenon by using the movement mechanism unit.

4. The movement function improvement device according to claim 1,
wherein the periarticular detection unit detects, as the periarticular physical quantities, an absolute angle, a rotation angle, an angular velocity, angular acceleration, and a driving torque between a rotator-side frame and a stator-side frame of the drive unit for the movement mechanism unit.

5. A movement function improvement method in a state where a movement mechanism unit having a drive unit to be driven actively or passively in conjunction with body movements of a subject is used in a manner united with the subject,
the movement function improvement method comprising:
a voluntary control step of controlling the drive unit to cause the subject's intention to move to be reflected in a kinetic phenomenon based on a biopotential signal, which indicates changes of an ionic current transmitted from a cranial nervous system to a muscular system of the subject and appears on a skin surface, and periarticular physical quantities which are caused by body movements of the subject and detected based on an output signal from the drive unit;
an autonomous control step of storing respective reference parameters of phases which are a series of minimum movement units constituting the subject's movement patterns classified as tasks, estimating a phase of the subject's task by comparing the periarticular physical quantities with the reference parameters stored in the data storage unit, and controlling the drive unit to generate motive power according to the phase; and
a synthetic control step of storing a control ratio of the voluntary control step and the autonomous control step, which is set for each phase of each task, in the data storage unit and synthesizing control states by the voluntary control step and the autonomous control step so as to achieve the control ratio according to the relevant phase,
wherein in the synthetic control step,
physical impedance of an entire system, which is composed of the entire device and the subject, is compensated for based on the periarticular physical quantities in accordance with physical properties and gravity of the entire system including human body properties of the subject; and
a difference between the subject's intention to move and the kinetic phenomenon is corrected by a movement command from the cranial nervous system and, at the same time, the synthesized control state is feedback-adjusted on the basis of biofeedback loops interactively promoted between the subject's body and the movement mechanism unit in order to minimize the difference between the subject's intention to move and the kinetic phenomenon.

6. The movement function improvement method according to claim 4,
comprising a walking state recognition step of recognizing a walking state of the subject based on the control state by the synthetic control step and a gravity center position of the subject detected based on a load change on foot sole surfaces of the subject,
wherein in the voluntary control unit step, the drive unit is controlled to cause the subject's intention to move to be reflected in the kinetic phenomenon on the basis of the biopotential signal, the periarticular physical quantities, and the walking state recognized by walking state recognition step.

7. The movement function improvement method according to claim 5,
wherein when forming the biofeedback loops, a minimum motion control unit for realizing voluntary movements caused by the subject's intention is set as a minimum voluntary motor control unit composed of the cranial nervous system, synaptic connections, and the muscular system; and
wherein the biofeedback loops are established for each minimum voluntary motor control unit that forms body movements which are performed cooperatively to realize the kinetic phenomenon by using the movement mechanism unit.

8. The movement function improvement method according to claim 5,
wherein an absolute angle, a rotation angle, an angular velocity, angular acceleration, and a driving torque between a rotator-side frame and a stator-side frame of the drive unit for the movement mechanism unit are detected as the periarticular physical quantities.
